(19) United States
(12) Patent Application Publication
(10) Pub. No.: US 2023/0214465 A1
(43) Pub. Date: Jul. 6, 2023

(54) MEDICAL INFORMATION CONTROL APPARATUS, SYSTEM, METHOD, AND NON-TRANSITORY COMPUTER READABLE MEDIUM STORING PROGRAM

(71) Applicant: NEC Corporation, Minato-ku, Tokyo (JP)

(72) Inventor: Nobuaki KAWASE, Tokyo (JP)

(73) Assignee: NEC Corporation, Minato-ku, Tokyo (JP)

(21) Appl. No.: 17/927,236

(22) PCT Filed: May 28, 2020
(86) PCT No.: PCT/JP2020/021227

§ 371 Date: Nov. 22, 2022

(51) Int. Cl. G06F 21/32; G16H 10/60

(52) U.S. Cl. CPC G06F 21/32; G16H 10/60

(57) ABSTRACT

A medical information control apparatus includes at least one memory storing instructions, and at least one processor. The at least one processor is configured to execute the instructions to acquire image data including a face image of a user captured by a predetermined image-capturing apparatus, cause an authentication apparatus that stores face feature information of the user to perform facial authentication for the image data, acquire, when the facial authentication has been successful, attribute information of the user related to the facial authentication from a storage apparatus that stores the attribute information of the user including information on at least one of a medical practice, a medical examination, and a drug preparation for the user, output the attribute information associated with the user to a predetermined display terminal that corresponds to the image-capturing apparatus.

### TECHNICAL FIELD

The present invention relates to a medical information control apparatus, a system, a method, and a program.

### BACKGROUND ART

Various kinds of systems that deal with medical information have been developed.

In a system disclosed in Patent Literature 1, for example, when non-prescription medical services or products are purchased by a method such as electronic money transaction, a facial recognition system is utilized for verification of the subject's identity, request, or receipt of the product or service requested.

In a medical information system disclosed in Patent Literature 2, a medical institution information processing apparatus requires authentication of a patient and a doctor and a home information processing apparatus requires authentication of the patient. Biometric information for identity authentication used here is, for example, biometric identification data for performing authentication by fingerprints. This literature discloses that the biometric information for identity authentication may include, besides the aforementioned data, the patient's face image.

### CITATION LIST

### Patent Literature

[Patent Literature 1] International Patent Publication No. WO 2015/073375
[Patent Literature 2] Japanese Unexamined Patent Application Publication No.

### SUMMARY OF INVENTION

### Technical Problem

The patient who has had his/her identity confirmed in a medical institution or the like often needs to provide various kinds of other information as well. According to the aforementioned techniques, however, even after the patient's identity has been successfully confirmed, if the language this patient uses is different from the languages that the staff members etc. in the medical institution use or the patient's level of consciousness is too low to communicate with the staff members etc., it is difficult or impossible to exchange information. According to the aforementioned techniques, there are other cases where patients may not be able to appropriately provide information desired by medical staff in the medical institution or the like.

The present disclosure has been made in order to solve the aforementioned problem, and an object of the present disclosure is to provide a medical information control apparatus, a system, a method, and a program for providing desired medical information for a medical institution or the like using a facial recognition technique.

### Solution to Problem

A medical information control apparatus according to one example embodiment of the present disclosure includes image data acquisition means, authentication control means, attribute information acquisition means, and output means. The image data acquisition means acquires image data including a face image of a user captured by a predetermined image-capturing apparatus. The authentication control means causes an authentication apparatus that stores face feature information of the user to perform facial authentication for the image data. The attribute information acquisition means acquires, when the facial authentication has been successful, attribute information of the user related to the facial authentication from a storage apparatus that stores the attribute information of the user including information on at least one of a medical practice, a medical examination, and a drug preparation for the user. The output means outputs the attribute information associated with the user to a predetermined display terminal that corresponds to the image-capturing apparatus.

In a medical information control method according to one example embodiment of the present disclosure, a computer performs the following steps. The computer acquires image data including a face image of a user captured by a predetermined image-capturing apparatus. The computer causes an authentication apparatus that stores face feature information of the user to perform facial authentication for the image data. The computer acquires, when the facial authentication has been successful, attribute information from a storage apparatus that stores the user and the attribute information of the user in association with each other. The computer outputs the attribute information associated with the user to a predetermined display terminal.

A program according to one example embodiment of the present disclosure causes a computer to execute the following steps. The computer acquires image data including a face image of a user captured by a predetermined image-capturing apparatus. The computer causes an authentication apparatus that stores face feature information of the user to perform facial authentication for the image data. The computer acquires, when the facial authentication has been successful, attribute information from a storage apparatus that stores the user and the attribute information of the user in association with each other. The computer outputs the attribute information associated with the user to a predetermined display terminal.

### Advantageous Effects of Invention

According to the present disclosure, it is possible to provide a medical information control apparatus, a system, a method, and a program for providing desired medical information for a medical institution or the like using a facial recognition technique.

### BRIEF DESCRIPTION OF DRAWINGS

FIG. 1 is a block diagram showing a configuration of a medical information control apparatus according to a first example embodiment;

FIG. 2 is a flowchart showing a medical information control method according to the first example embodiment;

FIG. 3 is a block diagram showing a configuration of a medical information control apparatus according to a second example embodiment;

FIG. 4 is a flowchart showing a first example of a medical information control method according to the second example embodiment;

FIG. 5 is a flowchart showing a second example of the medical information control method according to the second example embodiment;

FIG. 6 is a block diagram showing a configuration of a medical information control apparatus according to a third example embodiment;

FIG. 7 is a flowchart showing a medical information control method according to the third example embodiment;

FIG. 8 is a block diagram showing a configuration of a medical information control system according to a fourth example embodiment;

FIG. 9 is a block diagram showing a configuration of an authentication apparatus;

FIG. 10 is a flowchart showing a flow of face feature information registration processing;

FIG. 11 is a flowchart showing a flow of facial authentication processing;

FIG. 12 is a block diagram showing a configuration of a facial authentication unit;

FIG. 13 is a block diagram showing a configuration of a user terminal;

FIG. 14 is a block diagram showing a configuration of a medical information control apparatus; and

FIG. 15 is a diagram showing an example of a screen displayed on a facial authentication unit.

### EXAMPLE EMBODIMENT

In the following description, with reference to the drawings, example embodiments of the present disclosure will be described in detail. Throughout the drawings, the same or corresponding components are denoted by the same reference symbols and overlapping descriptions will be omitted as necessary for the sake of clarification of the description.

### First Example Embodiment

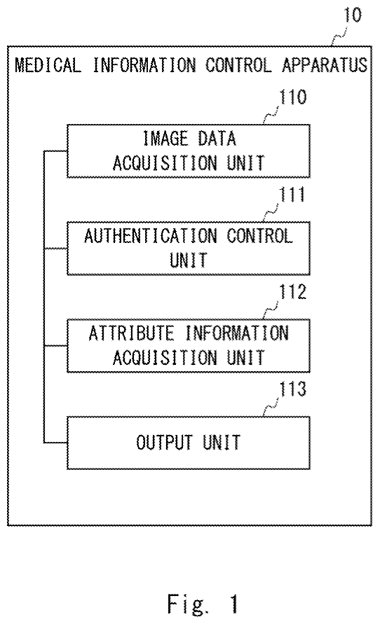

Referring to FIG. 1, a configuration of a medical information control apparatus will be described. FIG. 1 is a block diagram showing a configuration of a medical information control apparatus 10 according to a first example embodiment. The medical information control apparatus 10 performs facial authentication for a user such as a patient who uses a facility such as a medical institution, a clinic institution, or a pharmacy, and thus outputs information on at least one of a medical practice, a medical examination, and a drug preparation for the user. In the following description, the facility such as the medical institution, the clinic institution, or the pharmacy may be simply referred to as a “medical institution or the like”. The medical information control apparatus 10 is connected to a communication network (hereinafter the communication network will be simply referred to as a network) and performs various kinds of communication with an external apparatus via the connected network.

Specifically, the medical information control apparatus 10 is connected to an image-capturing apparatus, an authentication apparatus, a storage apparatus, and a display terminal via the network. The image-capturing apparatus shoots the user's face and supplies image data of the captured image to the medical information control apparatus 10. The authentication apparatus receives the image data from the medical information control apparatus 10 and performs facial authentication in accordance with the received image data. The storage apparatus stores attribute information of the user and provides the attribute information of the user related to the facial authentication for the medical information control apparatus 10. The display terminal receives the attribute information of the user related to the facial authentication from the medical information control apparatus 10 and displays the received attribute information. The network may either be a wired network or a wireless network. Further, any type of communication protocol may be used. The medical information control apparatus 10 includes, as main components, an image data acquisition unit 110, an authentication control unit 111, an attribute information acquisition unit 102, and an output unit 113.

The image data acquisition unit 110 acquires the image data including the face image of the user captured by the predetermined image-capturing apparatus. The authentication control unit 111 causes the authentication apparatus that stores face feature information of the user to perform facial authentication regarding the image data.

The attribute information acquisition unit 102 acquires, when the facial authentication has been successful, the attribute information of the user related to the facial authentication from the storage apparatus that stores the attribute information of the user. The attribute information of the user, which is information indicating the user's attributes, includes information on at least one of the medical practice, the medical examination, and the drug preparation for the user. The attribute information of the user may include, for example, besides user profile information such as the user's name, sex, nationality, the language the user uses, and the user's address, blood type, medical record, whether or not the user is pregnant, or kinds of medicines that this user is taking. The attribute information of the user may further include information regarding any allergy that the user has, the medical record (medical history) of the user, information regarding chronic diseases that the user has, information indicating whether or not the user would like to have generic drugs, or information on whether or not the user wishes to donate organs. The attribute information of the user may further include information regarding the medical insurance or the travel accident insurance purchased by the user. The attribute information of the user may further include the user's emergency contact information, if the user is travelling, the itinerary, information on the place where the user is visiting or staying, or information regarding payment means that the user can use. In the following description, the medical practice, the medical examination, the drug preparation and the like for the user may be referred to as practice performed by medical staff for the user.

The output unit 113 outputs the attribute information associated with the user to a predetermined display terminal that corresponds to the image-capturing apparatus. Cases in which the predetermined display terminal corresponds to the image-capturing apparatus include, for example, a case in which the image-capturing apparatus and the display terminal are provided in the same facility, a case in which the administrator who manages the image-capturing apparatus is the same as the administrator who manages the display terminal, or a case in which the administrator who manages the image-capturing apparatus and the administrator who manages the display terminal perform a predetermined operation in collaboration with each other. More specifically, the place where the image-capturing apparatus or the display terminal is provided is, for example, a facility (a medical facility, etc.) such as a medical institution, a clinic institution, or a pharmacy. The aforementioned attribute information is sent to the medical staff, whereby the medical information control apparatus 10 helps to support the practice that the medical staff provides for the user.

The image-capturing apparatus and the display terminal may each be a mobile terminal (a user terminal) that the user has. In this case, the user terminal is, for example, a mobile phone, a portable PC, a tablet terminal, a smartphone or the like. In this case, the user captures his/her face image using his/her user terminal. The user terminal supplies image data to the medical information control apparatus 10. Then the medical information control apparatus 10 receives the image data supplied from the user terminal. When the authentication is successful, the medical information control apparatus 10 outputs the attribute information of the user to the user terminal. Accordingly, the user who uses the medical information control apparatus 10 is able to present the attribute information displayed on the user terminal for the medical staff.

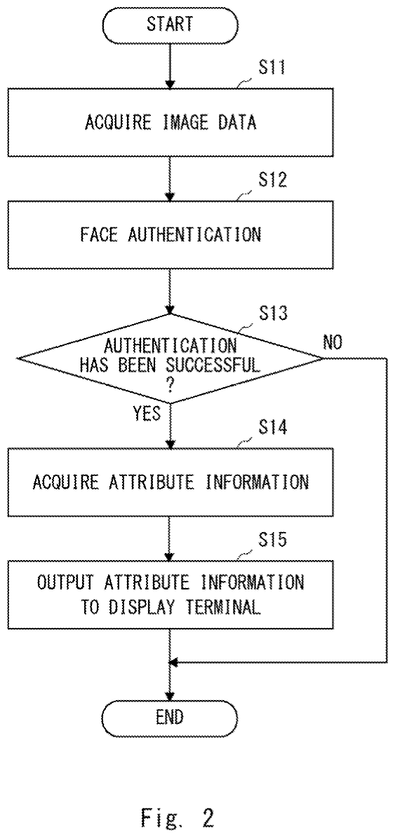

Referring next to FIG. 2, processing performed by the medical information control apparatus 10 will be described. FIG. 2 is a flowchart showing a medical information control method performed by the medical information control apparatus 10 according to the first example embodiment.

First, the medical information control apparatus 10 acquires the image data from the image-capturing apparatus via the network (Step S11). In this case, the medical information control apparatus 10 may request the image-capturing apparatus to transmit the image data or may receive the image data transmitted from the image-capturing apparatus for each predetermined period.

Next, the medical information control apparatus 10 causes the authentication apparatus to perform the facial authentication (Step S12). More specifically, the medical information control apparatus 10 supplies the image data received from the image-capturing apparatus to the authentication apparatus and requests the authentication apparatus to perform authentication. The authentication apparatus stores identification information of a plurality of users in association with face feature information of the plurality of users. The authentication apparatus extracts, for example, predetermined features from the face image of the user included in the image data and associates the extracted features with the identification information of the user. Further, when the authentication has been successful, the authentication apparatus supplies the identification information of the user associated with the received image data to the medical information control apparatus 10.

Next, the medical information control apparatus 10 determines whether or not the authentication has been successful (Step S13). Whether or not the authentication has been successful is determined, for example, by a signal sent from the authentication apparatus. When, for example, the signal indicating that the authentication has been successful is not acquired from the authentication apparatus within a predetermined period after a request signal for performing authentication has been transmitted, the medical information control apparatus 10 does not determine that the authentication has been successful (Step S13: NO). In this case, the medical information control apparatus 10 ends the processing.

On the other hand, when, for example, the signal indicating that the authentication has been successful has been acquired from the authentication apparatus within a predetermined period after the request signal for performing authentication has been transmitted, the medical information control apparatus 10 determines that the authentication has been successful (Step S13: YES). In this case, the medical information control apparatus 10 acquires the attribute information of the user related to the successful authentication from the storage apparatus (Step S14). Next, the medical information control apparatus 10 outputs the acquired attribute information to the display terminal (Step S15), and the processing is ended.

The medical information control apparatus 10 according to the first example embodiment has been described above. Note that the medical information control apparatus 10 includes, as components that are not shown, a processor and a storage apparatus. The storage apparatus included in the medical information control apparatus 10 includes a storage apparatus including a non-volatile memory such as a flash memory or a Solid State Drive (SSD). In this case, the storage apparatus included in the medical information control apparatus 10, which stores the attribute information, may read the attribute information of the user according to the successful facial authentication from the aforementioned storage apparatus.

The storage apparatus that the medical information control apparatus 10 includes stores a computer program (hereinafter it may also be simply referred to as a program) for executing the medical information control method according to this example embodiment. Further, the processor loads a computer program into a memory from a storage apparatus and executes this program.

Each of the components that the medical information control apparatus 10 includes may be implemented by dedicated hardware. Further, some or all of the components may each be implemented by general-purpose or dedicated circuitry, processor, or a combination of them. They may be configured using a single chip, or a plurality of chips connected through a bus. Some or all of the components of each apparatus may be implemented by a combination of the above-described circuitry, etc. and a program. Further, a Central Processing Unit (CPU), a Graphics Processing Unit (GPU), a field-programmable gate array (FPGA) and so on may be used as a processor.

Further, when some or all of the components of the medical information control apparatus 10 are implemented by a plurality of information processing apparatuses, circuits, or the like, the plurality of information processing apparatuses, the circuits, or the like may be disposed in one place in a centralized manner or arranged in a distributed manner. For example, the information processing apparatuses, the circuits, and the like may be implemented as a client-server system, a cloud computing system or the like, or a configuration in which the apparatuses or the like are connected to each other through a communication network. Alternatively, the functions of the medical information control apparatus 10 may be provided in the form of Software as a Service (SaaS).

The aforementioned medical information control apparatus 10 outputs the attribute information of the user to the medical institution or the like by performing the facial authentication of the user. This eliminates the need for the user to perform an operation of providing information for the medical institution or the like. Further, even in a case in which the language that the user uses is different from languages that staff members in the medical institution or the like use or in a situation in which it is difficult or impossible for the user to communicate with others, the medical information control apparatus 10 is able to suitably output the attribute information of the user. Further, the medical information control apparatus 10 performs the facial authentication, and thus outputs information definitely associated with the user himself/herself. It is therefore possible to reduce fraudulent authentication. Further, the medical information control apparatus 10 outputs, for example, information on the user's travel accident insurance to the display terminal. In this case, even in a situation in which it is difficult or impossible for the user to communicate with others in the travel destination, the medical institution or the like can access the travel accident insurance purchased by the user from the attribute information related to the facial authentication. Therefore, the medical institution or the like is able to receive payment for medical expenses, etc. using the output attribute information. That is, according to this example embodiment, it is possible to provide the medical information control apparatus, the system, the method, and the program for providing desired medical information for a medical institution or the like using the facial recognition technique.

### Second Example Embodiment

Next, a second example embodiment will be described. A medical information control apparatus according to the second example embodiment is different from that of the first example embodiment in that the medical information control apparatus according to the second example embodiment includes a receiving unit and a storage unit. Further, the aspect in the acquisition or the output of the attribute information in the medical information control apparatus according to the second example embodiment is different from that according to the first example embodiment.

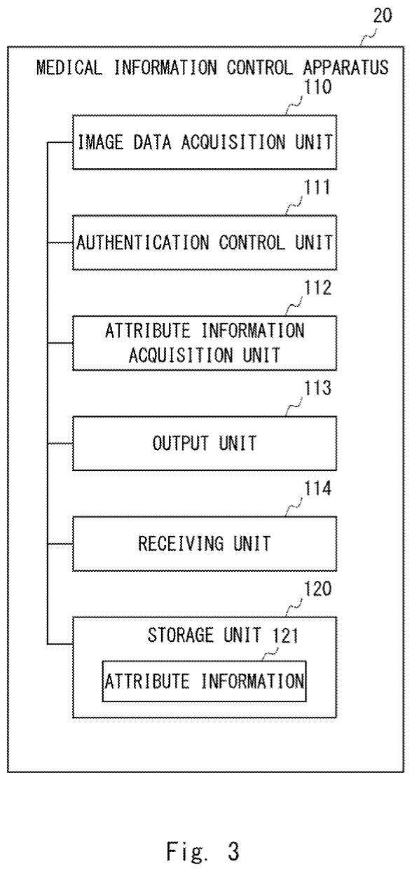

FIG. 3 is a block diagram showing a configuration of the medical information control apparatus according to the second example embodiment. A medical information control apparatus 20 shown in FIG. 3 mainly includes an image data acquisition unit 110, an authentication control unit 111, an attribute information acquisition unit 102, an output unit 113, a receiving unit 114, and a storage unit 120.

The receiving unit 114 receives an input of a predetermined signal from a display terminal via a network. The predetermined signal may be, for example, a condition for extracting the attribute information. Further, the predetermined signal may be, for example, an inquiry signal related to the medical practice, the medical examination, the drug preparation and the like for the user.

The storage unit 120, which is a storage apparatus including a non-volatile memory, stores at least attribute information 121 of the user. Besides the attribute information 121 being associated with the user's identification information, the attribute information 121 includes index information associated with each of the pieces of the attribute information. The index information is associating information for enabling a plurality of pieces of attribute information to be extracted or retrieved for each layer.

For example, as the information regarding the user's blood type, user-specific attribute information such as “type A” may be associated with index information, which is “blood type”. Further, for example, the attribute information regarding whether or not the user would like to have generic drugs may be associated with index information, which is “preparation of medicines”. Since the attribute information includes the index information, the medical information control apparatus 20 is able to output the attribute information by various aspects. The medical information control apparatus 20 is able to customize, for example, the attribute information to be output to the display terminal using the index information.

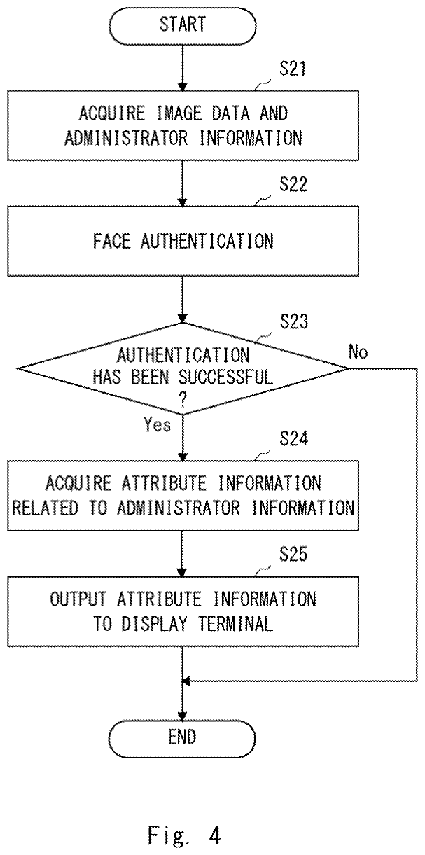

Next, processing performed by the medical information control apparatus 20 will be described. FIG. 4 is a flowchart showing a first example of the medical information control method according to the second example embodiment. The processing when the attribute information is acquired in the flowchart shown in FIG. 4 is different from that in the flowchart according to the first example embodiment. In the following flowcharts, the description of processing that is similar to that in the aforementioned flowchart will be omitted as appropriate.

First, the image data acquisition unit 110 of the medical information control apparatus 20 acquires the image data and administrator information from the image-capturing apparatus via the network (Step S21). The administrator information is information regarding the administrator who manages the image-capturing apparatus. The administrator information includes, for example, category information such as a hospital, a clinic, or a pharmacy. The administrator information is supplied to the medical information control apparatus 20 along with the image data that the image-capturing apparatus transmits.

Next, the medical information control apparatus 20 causes the authentication apparatus to perform the facial authentication (Step S22). Next, the medical information control apparatus 20 determines whether or not the authentication has been successful (Step S23). When it is not determined that the authentication has been successful (Step S23: NO), the medical information control apparatus 20 ends the processing.

On the other hand, when it is determined that the authentication has been successful (Step S23: YES), the medical information control apparatus 20 acquires, of the attribute information of the user related to the successful authentication, the attribute information related to the administrator information acquired in Step S21 from the storage apparatus (Step S24). Next, the medical information control apparatus 20 outputs the acquired attribute information to the display terminal (Step S25), and the processing is ended.

The flowchart shown in FIG. 4 has been described above. The image data acquisition unit 110 acquires the administrator information regarding the administrator of the image-capturing apparatus along with the image data, and the attribute information acquisition unit 102 acquires the attribute information associated with the administrator information from the storage apparatus. According to the flowchart shown in FIG. 4, when, for example, the administrator of the image-capturing apparatus is a pharmacy, the medical information control apparatus 20 outputs the attribute information related to the pharmacy to the display terminal.

Note that, in the medical information control method shown in FIG. 4, the image data acquisition unit 110 may also acquire the administrator information regarding the administrator of the image-capturing apparatus and the attribute information acquisition unit 102 may output the attribute information based on the administrator information to the display terminal. In this case, the attribute information acquisition unit 102 may acquire the attribute information of the user related to the facial authentication regardless of the above administrator information, then extract the attribute information based on the aforementioned administrator information from the acquired attribute information, and further output the extracted attribute information. Further, in the medical information control apparatus 20 that executes the method shown in FIG. 4, the receiving unit 114, not the image data acquisition unit 110, may receive the administrator information.

According to the aforementioned configuration, the medical information control apparatus 20 is able to suppress provision of information that is unnecessary for an administrator and is able to suitably deal with user's information from the viewpoint of protecting privacy.

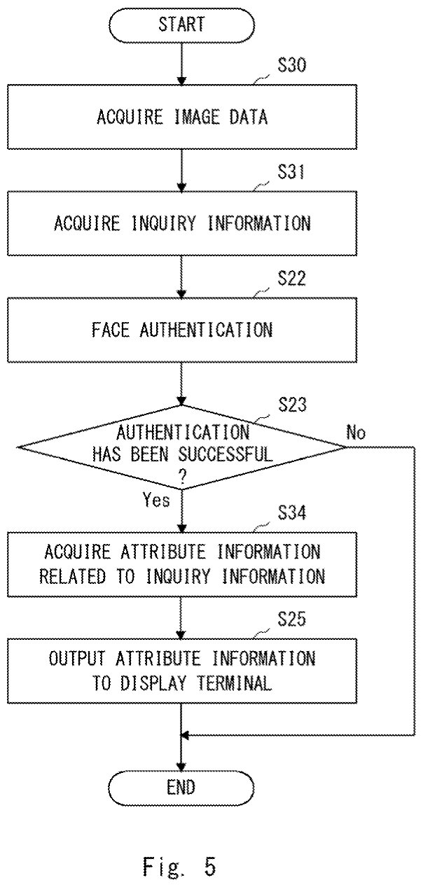

Next, another example of processing performed by the medical information control apparatus 20 will be described. FIG. 5 is a flowchart showing a second example of the medical information control method according to the second example embodiment. The flowchart shown in FIG. 5 is different from the flowchart according to the first example embodiment in that the processing of the receiving unit 114 is added in the flowchart shown in FIG. 5.

First, in the medical information control apparatus 20, the image data acquisition unit 110 acquires the image data from the image-capturing apparatus (Step S30). Then, in the medical information control apparatus 20, the receiving unit 114 acquires inquiry information from the display terminal (Step S31). The inquiry information, which is information desired by the administrator of the display terminal, is information included in the attribute information of the user. When, for example, the administrator of the display terminal is a pharmacy, the administrator is able to inquire whether or not the user related to the facial authentication would like to have generic drugs.

Next, the medical information control apparatus 20 causes the authentication apparatus to perform the facial authentication (Step S22). Next, the medical information control apparatus 20 determines whether or not the authentication has been successful (Step S23). When it is not determined that the authentication has been successful (Step S23: NO), the medical information control apparatus 20 ends the processing.

On the other hand, when it is determined that the authentication has been successful (Step S23: YES), the medical information control apparatus 20 acquires, of the attribute information of the user related to the successful authentication, the attribute information related to the inquiry information acquired in Step S31 from the storage apparatus (Step S34). Next, the medical information control apparatus 20 outputs the acquired attribute information to the display terminal (Step S25) and the processing is ended. In the aforementioned example, for example, the medical information control apparatus 20 outputs, when information indicating whether or not the user would like to have generic drugs has been acquired, information regarding whether the user would like to have generic drugs to the display terminal from the attribute information of the user related to the facial authentication.

The flowchart in FIG. 5 has been described above. In the aforementioned flowchart, Step S31 may be performed anywhere before Step S34. As described above, the medical information control apparatus 20 according to this example embodiment further includes the receiving unit 114 that receives the input of the predetermined signal from the display terminal and the output unit 113 outputs the attribute information in accordance with the above signal to the display terminal. More specifically, for example, the receiving unit 114 receives a condition for extracting the attribute information. Then, the output unit 113 extracts predetermined information included in the attribute information in accordance with the above signal and outputs the extracted information to the display terminal. The receiving unit 114 may receive an inquiry related to at least one of the medical practice, the medical examination, and the drug preparation for the user. In this case, the output unit 113 extracts the attribute information, which is a response to the inquiry, from the attribute information and outputs the extracted attribute information. As discussed above, with the medical information control method shown in FIG. 5, the medical information control apparatus 20 is able to suitably provide information desired by the administrator.

In the aforementioned medical information control apparatus 20, the receiving unit 114 may receive a message to be sent to the user. In this case, the output unit 113 sends a message to the user based on an access method included in the attribute information. When, for example, the user's email address is included in the attribute information, the medical information control apparatus 20 is able to transmit a message for the user to the user's email address.

Further, the aforementioned medical information control apparatus 20 may further include a third party information acquisition unit that acquires third party information related to the message. In this case, the output unit 113 notifies the user of the message and the third party information related to the message. The third party is, for example, a payment service provider who provides payment means when the fee for the practice performed by the medical staff is paid. In this case, the third party information acquisition unit acquires information on promotions or the like provided from the payment service provider. The output unit 113 is able to receive the information on the promotions offered by the payment service provider when the fee is paid to the medical institution or the like.

Further, the aforementioned medical information control apparatus 20 may notify the user of information on the medical institution or the like contracted by a medical insurance company with which the user has a contract as third party information related to the message. When, for example, it becomes difficult or impossible for the user to communicate with others due to a sudden accident or illness, the medical information control apparatus 20 is able to output the medical institution or the like to which the user is transported to the display terminal from the attribute information of the user.

The aforementioned medical information control apparatus 20 outputs the attribute information of the user to the medical institution or the like by performing the facial authentication of the user. Accordingly, according to the second example embodiment, it becomes possible to provide the medical information control apparatus, the system, the method, and the program for providing desired medical information or the like for the medical institution or the like using the facial recognition technique.

### Third Example Embodiment

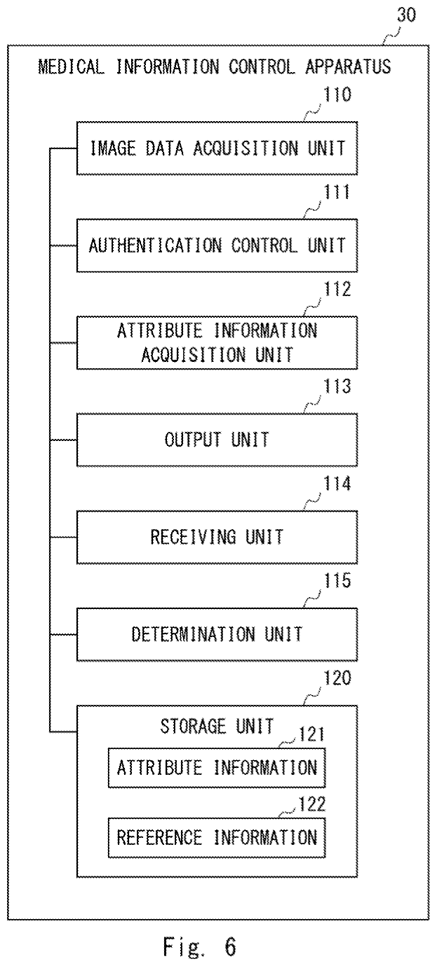

Next, a third example embodiment will be described. A medical information control apparatus according to the third example embodiment is different from those of the aforementioned example embodiments in that the medical information control apparatus according to the third example embodiment includes a determination unit. Further, the medical information control apparatus according to the third example embodiment is different from that of the first example embodiment in that the storage unit further stores, besides the attribute information, reference information in the third example embodiment. FIG. 6 is a block diagram showing a configuration of the medical information control apparatus according to the third example embodiment. A medical information control apparatus 30 shown in FIG. 6 mainly includes an image data acquisition unit 110, an authentication control unit 111, an attribute information acquisition unit 102, an output unit 113, a receiving unit 114, a determination unit 115, and a storage unit 120.

The receiving unit 114 according to this example embodiment receives an inquiry about suitability of a medical practice, a medical examination, or a drug preparation for a user related to facial authentication. More specifically, the receiving unit 114 receives, for example, inquiry information from the display terminal. The inquiry information includes information regarding the medical practice, the medical examination, or drugs to be prepared for the user related to the facial authentication, and a request for determining the suitability of the above information.

The determination unit 115 refers to each of the inquiry information received by the receiving unit 114, and the attribute information and the reference information stored in the storage unit 120, generates results of a determination in response to the inquiry, and supplies the generated results of the determination to the output unit 113. The output unit 113 outputs the results of the determination in response to the above inquiry to the display terminal.

The reference information stored in the storage unit 120 is reference information for determining suitability of a combination of information regarding the medical practice, the medical examination, or drugs to be prepared for the user related to the facial authentication with the attribute information of the user, the reference information being included in the inquiry information.

The reference information may include, for example, information on a combination of medicines. In this case, the attribute information includes information on medications the user is currently taking. In this case, after the receiving unit 114 receives an inquiry regarding candidate medicines that the user might take, the determination unit 115 refers to each of the reference information and the attribute information. The determination unit 115 determines whether or not a combination of the candidate for the medicines with the medications the user is currently taking is included in the information on the combination of medicines if the user takes the candidate medicines according to the inquiry. Note that the information on the combination of medicines may include information regarding suitability of the combination of medicines or information on the acceptability of the combination.

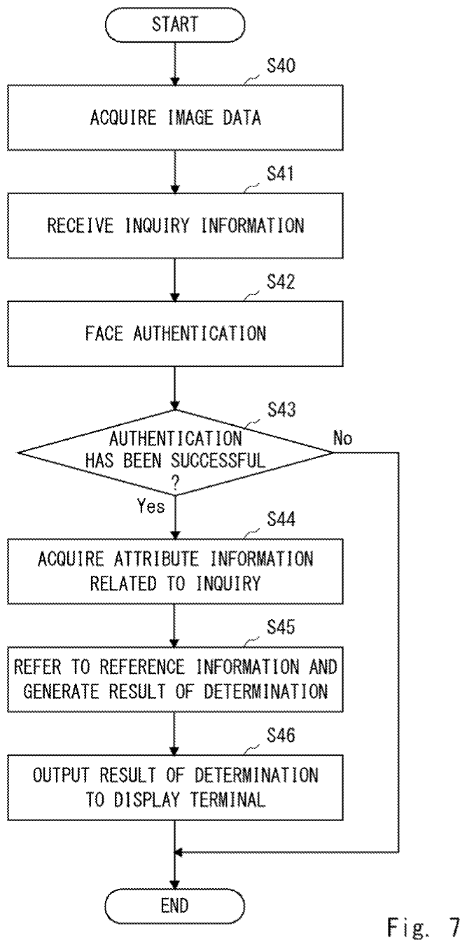

Referring next to FIG. 7, processing executed by the medical information control apparatus 30 will be described. FIG. 7 is a flowchart showing a medical information control method according to the third example embodiment. The processing of receiving the inquiry and the processing regarding the determination in the flowchart shown in FIG. 7 are different from those in the flowchart shown in the aforementioned example embodiments.

First, in the medical information control apparatus 30, the image data acquisition unit 110 acquires the image data from the image-capturing apparatus (Step S40). Then, in the medical information control apparatus 30, the receiving unit 114 acquires the inquiry information from the display terminal (Step S41). This inquiry information includes a request for determination regarding the inquiry.

Next, the medical information control apparatus 30 causes the authentication apparatus to perform facial authentication (Step S42). Next, the medical information control apparatus 30 determines whether or not the authentication has been successful (Step S43). When it is not determined that the authentication has been successful (Step S43: NO), the medical information control apparatus 30 ends the processing.

On the other hand, when it is determined that the authentication has been successful (Step S43: YES), the medical information control apparatus 30 acquires, of the attribute information of the user related to the successful authentication, the attribute information related to the inquiry information acquired in Step S41 from the storage apparatus (Step S44). Next, the determination unit 115 of the medical information control apparatus 30 refers to reference information 122 stored in the storage unit 120 and generates the result of the determination (Step S45). Next, the output unit 113 outputs the result of the determination to the display terminal (Step S46), and the processing is ended.

The flowchart shown in FIG. 7 has been described above. In the aforementioned flowchart, Step S41 may be performed anywhere before Step S44. As described above, the medical information control apparatus 30 according to this example embodiment further includes a determination unit 115 that generates the result of the determination regarding the combination of the inquiry with the attribute information, the receiving unit 114 receives a determination request signal for requesting a determination, and the output unit 113 outputs the result of the determination that corresponds to the request to the display terminal.

According to the medical information control method shown in FIG. 7, the medical information control apparatus 30 is able to suitably provide information desired by the administrator. Further, the medical information control apparatus 30 is able to support the practice that the administrator provides for the user by outputting the result of the determination.

Note that the medical information control apparatus 30 may receive a signal for updating the attribute information from the receiving unit 114. In this case, the medical information control apparatus 30 may further include an update unit that updates the attribute information and causes the updated attribute information to be stored in the storage unit 120. When, for example, a pharmacy has provided medicines for the user, the administrator of the pharmacy who has provided the medicines supplies the name, the expiration date, etc. of the medicines provided for the user as update information to the medical information control apparatus 30 via the display terminal. Accordingly, the medical information control apparatus 30 is able to add information regarding medicines that the user has newly started taking to the attribute information.

As described above, according to the third example embodiment, it is possible to provide the medical information control apparatus, the system, the method, and the program for providing desired medical information for a medical institution or the like using the facial recognition technique.

### Fourth Example Embodiment

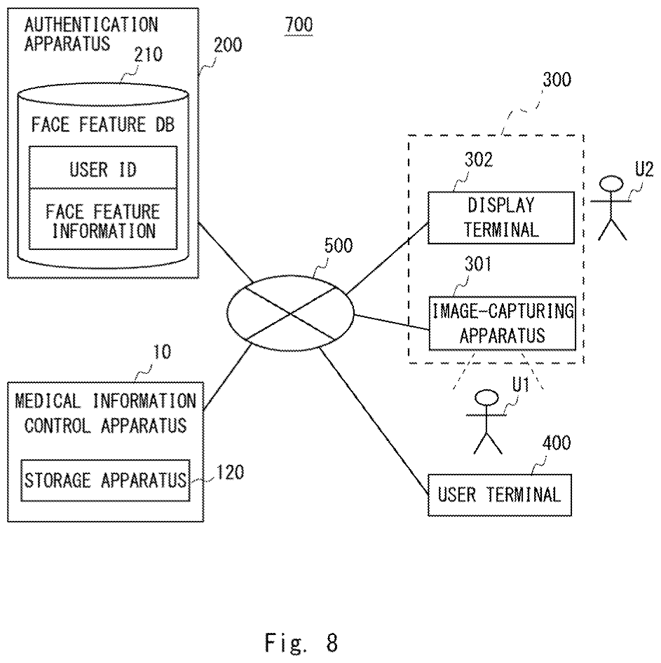

Next, a fourth example embodiment will be described. The fourth example embodiment is an example of a system including the medical information control apparatus 10 according to the aforementioned first example embodiment. FIG. 8 is a block diagram showing a configuration of a medical information control system 700 according to the fourth example embodiment. The medical information control system 700 includes a medical information control apparatus 10, an authentication apparatus 200, a facial authentication unit 300, and a user terminal 400. The medical information control apparatus 10, the authentication apparatus 200, the facial authentication unit 300, and the user terminal 400 are connected to one another via a network 500. It is assumed, in this example embodiment, that the medical information control apparatus 10 includes a storage unit 120. Further, explanations duplicating those of the first example embodiment will be omitted as appropriate.

The medical information control apparatus 10 is an information processing apparatus for outputting attribute information of a user U1 to the facial authentication unit 300 when authentication regarding a captured image of the user U1, who is a patient, has been successful. The medical information control apparatus 10 is, for example, a server apparatus achieved by a computer.

The authentication apparatus 200 is an apparatus that performs facial authentication. The authentication apparatus 200 includes a face feature DB 210. The face feature DB 210 is a face feature database that stores a user ID and face feature information of the user in association with each other. The face feature DB 210 is one example of the face feature information storage unit.

The facial authentication unit 300 includes the image-capturing apparatus (an image-capturing apparatus 301) and the display terminal (a display terminal 302) described in the first example embodiment. The facial authentication unit 300 is installed in a medical institution or the like. The facial authentication unit 300 is an information processing apparatus that shoots the face of the user U1 who has visited the medical institution or the like, transmits a facial authentication request including the captured image to the medical information control apparatus 10, and receives, when the facial authentication has been successful, the attribute information of the user U1 and displays the received attribute information. The facial authentication unit 300 is operated, for example, by a medical staff U2. Note that a plurality of the facial authentication units 300 may be installed in the medical institution or the like. Further, the image-capturing apparatus 301 and the display terminal 302 included in the facial authentication unit 300 may be integrated with each other or may be separated from each other.

The user terminal 400 is an information processing apparatus that is operated by a predetermined user. The user terminal 400 is, for example, a communication terminal such as a smartphone, a tablet, or a PC. The user terminal may perform processing that is similar to that performed in the facial authentication unit 300 or a part of the processing that is performed in the facial authentication unit 300.

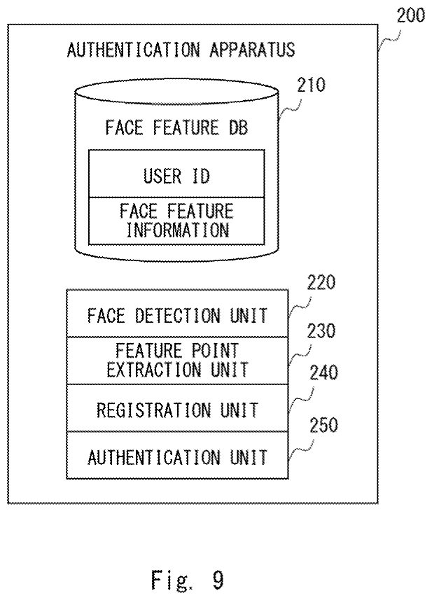

Referring next to FIG. 9, a configuration of the authentication apparatus 200 will be described in detail. FIG. 9 is a block diagram showing the configuration of the authentication apparatus 200. The authentication apparatus 200 includes a face feature DB 210, a face detection unit 220, a feature point extraction unit 230, a registration unit 240, and an authentication unit 250.

The face feature DB 210 is a face feature database that stores the user ID and the face feature information of the user in association with each other. The face detection unit 220 detects a face area that the captured image includes and outputs the detected face area to the feature point extraction unit 230. The feature point extraction unit 230 extracts feature points from the face area detected by the face detection unit 220 and outputs the face feature information to the registration unit 240. The face feature information is a set of the extracted feature points.

The registration unit 240 newly issues a user ID when the face feature information is registered. The registration unit 240 registers the issued user ID in association with the face feature information extracted from the registered image in the face feature DB 210. The authentication unit 250 compares the face feature information extracted from the face image with the face feature information in the face feature DB 210. When the two pieces of face feature information coincide with each other, the authentication unit 250 determines that the facial authentication has been successful. On the other hand, when the two pieces of face feature information do not coincide with each other, the authentication unit 250 determines that the facial authentication has failed. The authentication unit 250 sends information indicating whether or not the facial authentication has been successful back to the medical information control apparatus 10. Whether the two pieces of face feature information coincide with each other corresponds to the result indicating whether the authentication has been successful. Further, when the facial authentication has been successful, the authentication unit 250 specifies the user ID associated with the successful face feature information and sends results of the authentication including the specified user ID and information indicating that the authentication has been successful back to the medical information control apparatus 10.

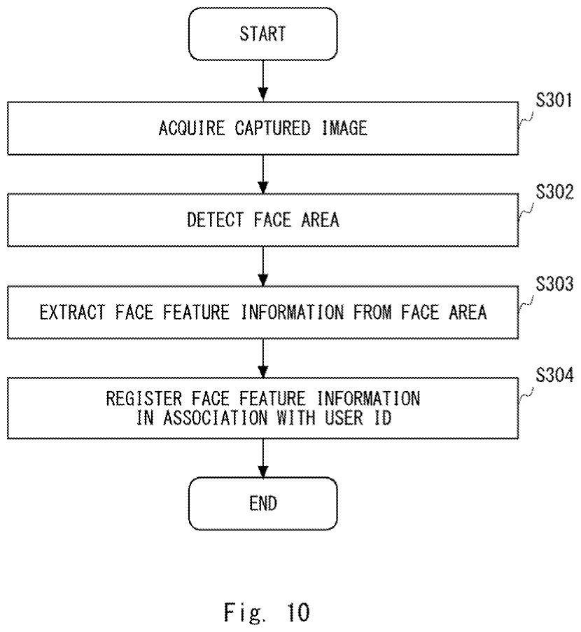

With reference to FIG. 10, an operation of the authentication apparatus 200 when the user ID and the face feature information are registered in the face feature DB 210 will be described. FIG. 10 is a flowchart showing a flow of face feature information registration processing.

When the face feature information is registered, a face information registration terminal (not shown) captures an image including the user's face as an image for registration and sends a face feature information registration request to the authentication apparatus 200 via the network 500. The face information registration terminal is, for example, an information processing apparatus such as a personal computer, a smartphone, or a tablet terminal. The face information registration terminal is, for example, the user terminal 400. The face feature information registration request includes an image for registration. First, the authentication apparatus 200 acquires the image for registration included in the face feature information registration request from the face information registration terminal via the network 500 (Step S301).

Next, the face detection unit 220 detects a face area that the image for registration includes (Step S302) and outputs the detected face area to the feature point extraction unit 230. Next, the feature point extraction unit 230 extracts feature points from the face area and outputs face feature information to the registration unit 240 (Step S303). Next, the registration unit 240 issues a user ID that corresponds to the output face feature information and registers this user ID in association with the face feature information in the face feature DB 210 (Step S304).

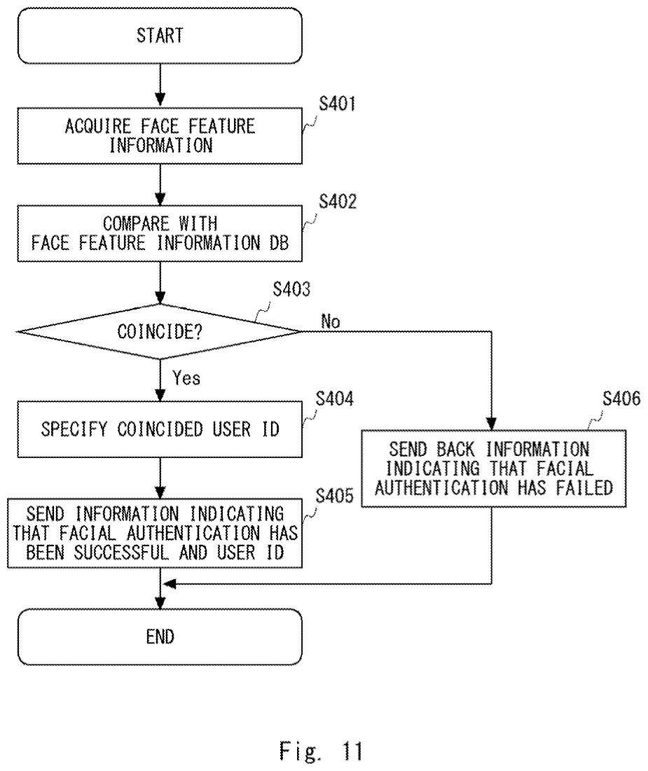

Referring to FIG. 11, an operation of the authentication apparatus 200 when the facial authentication is performed will be described. FIG. 11 is a flowchart showing a flow of facial authentication processing. When the facial authentication is performed, the facial authentication unit 300 captures an image including the user's face as an image for authentication and sends a facial authentication request to the medical information control apparatus 10 via the network 500. The facial authentication request includes an image for authentication. First, the medical information control apparatus 10 transmits the face area or the face feature information extracted from the image for authentication to the authentication apparatus 200. The feature point extraction unit 230 extracts the face feature information from the received face area or receives the face feature information, and thus acquires the face feature information (Step S401).

Next, the authentication unit 250 compares the acquired face feature information with the face feature information in the face feature DB 210 (Step S402). When the face feature information coincides with the face feature information in the face feature DB 210 (Step S403: YES), the authentication unit 250 specifies the ID of the user whose face feature information coincides with the face feature information in the face feature DB 210 (Step S404) and sends information indicating that the facial authentication has been successful and the specified user ID back to the medical information control apparatus 10 (Step S405). When there is no face feature information in the face feature DB 210 that coincides with the face feature information of the user (Step S403: NO), the authentication unit 250 sends information indicating that the facial authentication has failed back to the medical information control apparatus 10 (Step S406). The authentication apparatus 200 is able to estimate the age and sex of the user based on the face feature information. In Step S406, the authentication unit 250 sends information including the age and sex that have been estimated back to the medical information control apparatus 10.

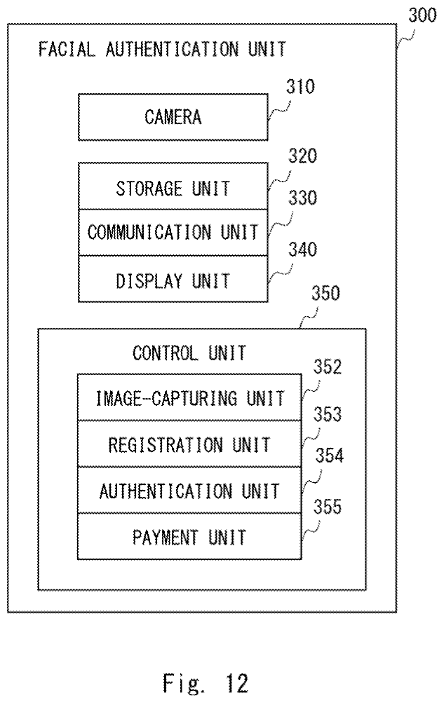

Referring next to FIG. 12, a configuration of the facial authentication unit 300 will be described in detail. The facial authentication unit 300 includes, as hardware components, an image-capturing apparatus such as a camera, a display apparatus, and a computer. The facial authentication unit 300 is an apparatus that sends a facial authentication request by facial authentication. The facial authentication unit 300 is, for example, but not limited to, a terminal that performs payment by facial authentication, a terminal that performs entry control by facial authentication, or digital signage that presents information in accordance with the facial authentication.

FIG. 12 is a block diagram showing a configuration of the facial authentication unit 300. The facial authentication unit 300 includes a camera 310, a storage unit 320, a communication unit 330, a display unit 340, and a control unit 350. The camera 310 is an image-capturing apparatus that captures images. That is, the camera 310 corresponds to the image-capturing apparatus 301 shown in FIG. 8.

The storage unit 320 is a storage apparatus that stores a program for implementing each of the functions of the facial authentication unit 300. The communication unit 330 is a communication interface with the network 500. The display unit 340 is a display apparatus that displays the result of the facial authentication, the medical information control information, or the like for the user. The control unit 350 controls hardware included in the facial authentication unit 300. The control unit 350 includes an image-capturing unit 352, a registration unit 353, an authentication unit 354, and a payment unit 355.

The image-capturing unit 352 sends an image-capturing request to the camera 310. The camera 310 captures an image including the user's face. The image captured by the camera 310 is used as an image for registration and an image for authentication. The registration unit 353 transmits a face feature information registration request to the authentication apparatus 200 via the network 500. The face feature information registration request includes an image for registration captured by the camera 310. The authentication unit 354 transmits a facial authentication request to the medical information control apparatus 10 via the network 500. The facial authentication request includes the image for authentication captured by the camera 310. The authentication unit 354 receives information indicating whether or not the facial authentication has been successful from the medical information control apparatus 10 and causes the display unit 340 to display the result of the facial authentication.

The payment unit 355 executes payment processing when the facial authentication has been successful. Note that the control unit 350 may include, in place of the payment unit 355, a recording unit (not shown). The recording unit may record the entry and exit by each user into and from the facility in which the facial authentication unit 300 is installed when the facial authentication has been successful. Note that the display terminal 302 shown in FIG. 8 may be the facial authentication unit 300 itself or may be a part other than the camera 310.

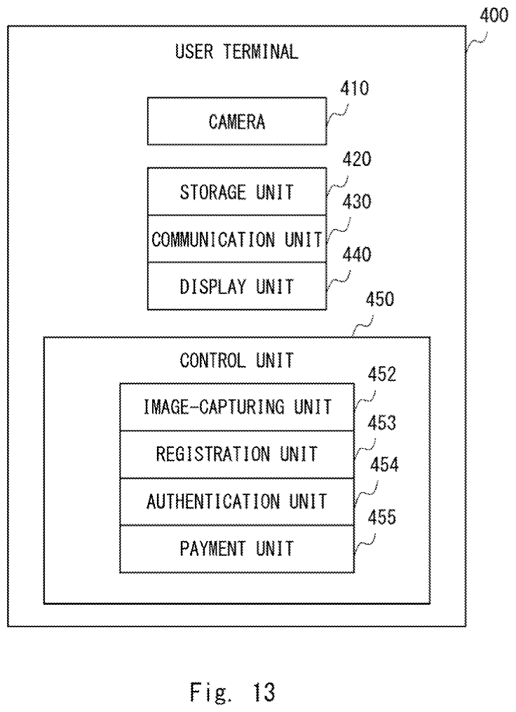

Referring next to FIG. 13, a configuration of the user terminal 400 will be described in detail. FIG. 13 is a block diagram showing a configuration of the user terminal 400. The user terminal 400 includes a camera 410, a storage unit 420, a communication unit 430, a display unit 440, and a control unit 450. The control unit 450 includes an image-capturing unit 452, a registration unit 453, an authentication unit 454, and a payment unit 455. Since the features of the components included in the user terminal 400 are similar to those of the components included in the facial authentication unit 300, the descriptions thereof will be omitted.

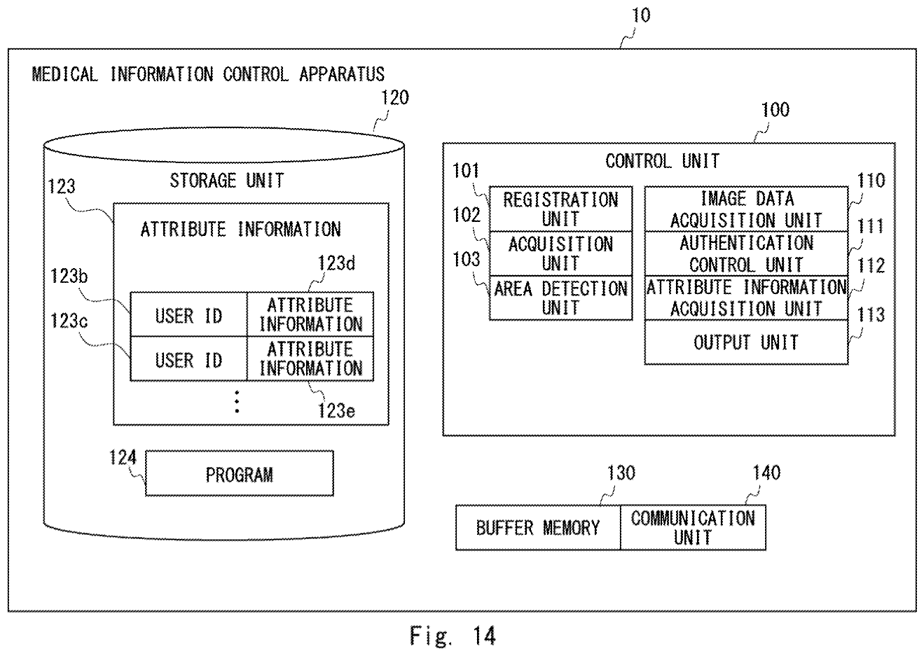

Referring next to FIG. 14, the configuration of the medical information control apparatus 10 will be described in detail. FIG. 14 is a block diagram showing the configuration of the medical information control apparatus 10. The medical information control apparatus 10 includes a control unit 100, a storage unit 120, a buffer memory 130, and a communication unit 140.

The storage unit 120 is a non-volatile storage apparatus that stores at least attribute information 123 and a program 124. The attribute information 123 is a set of pieces of user IDs of the respective users and attribute information associated with the respective users. The user ID, which is identification information by which it is possible to identify the user, is, for example, the name, the telephone number, the email address, or any identification number. Attribute information 123d and 123e are associated with user IDs 123b and 123c, respectively. That is, the identification information of the plurality of users and the attribute information of the plurality of respective users are associated with each other in the storage unit 120. The attribute information 123d and 123e are, for example, but not limited to, the sex, the age, hobbies, or the physical information. The program 124 is a computer program in which the medical information control processing according to this example embodiment is implemented.

The buffer memory 130, which is a storage area that temporarily stores the content of processing of the control unit 100, is, for example, a volatile storage apparatus such as a Random Access Memory (RAM). The communication unit 140 is an interface that performs communication between the medical information control apparatus 10 and an external apparatus or the like.

The control unit 100 includes a registration unit 101, an acquisition unit 102, an area detection unit 103, an image data acquisition unit 110, an authentication control unit 111, an attribute information acquisition unit 112, and an output unit 113. The control unit 100, which is a control apparatus that controls the operation of the medical information control apparatus 10, is, for example, a processor such as a CPU. The control unit 100 loads the program 124 into the buffer memory 130 from the storage unit 120 and executes the loaded program. Accordingly, the control unit 100 implements the functions as the registration unit 101, the acquisition unit 102, the area detection unit 103, the image data acquisition unit 110, the authentication control unit 111, the attribute information acquisition unit 112, and the output unit 113.

The registration unit 101 receives a registration request from the facial authentication unit 300 or the user terminal 400 via the network 500. The registration unit 101 issues a user ID that corresponds to the user ID included in the received registration request. The registration unit 101 generates the attribute information 123 in which the plurality of pieces of attribute information are associated with the issued user IDs and registers the generated attribute information 123 in the storage unit 120.

The acquisition unit 102 acquires image data from the image-capturing apparatus. The area detection unit 103 detects a face area from the image data. For example, the area detection unit 103 analyzes the image data and recognizes the face area of the user. The authentication control unit 111 causes, when the face area has been specified in the image data, the authentication apparatus 200 to perform facial authentication that uses face feature information for each of the specified face areas.

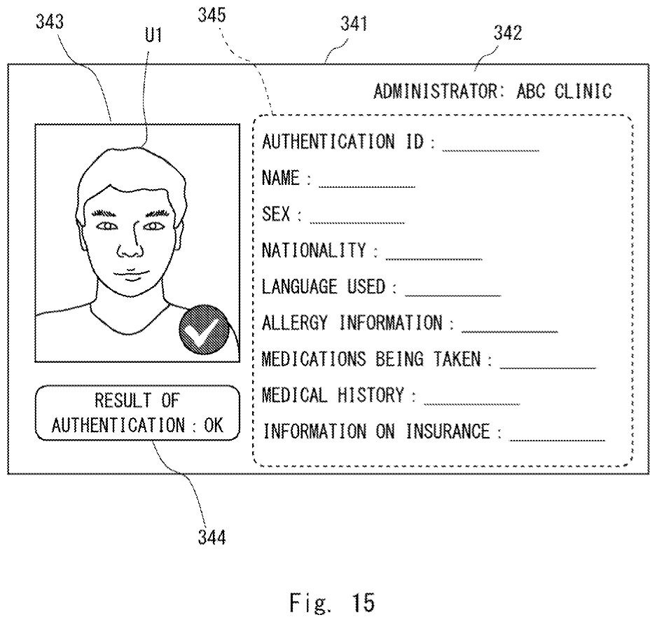

Referring to FIG. 15, a screen displayed by the facial authentication unit 300 (display terminal 302) will be described. FIG. 15 is a diagram showing an example of the screen displayed on the facial authentication unit. FIG. 15 shows a screen 341 displayed by the facial authentication unit 300. The screen 341 includes an information display 342 regarding the administrator, an image 343 related to the facial authentication of the user U1, a display 344 of the result of the facial authentication, and an attribute information display 345.

In the example shown in FIG. 15, the administrator of the facial authentication unit 300 is a clinic facility called ABC clinic. In the ABC clinic, the face of the user U1 who has come to the clinic has been taken by the image-capturing apparatus and an operation of requesting for attribute information has been performed. Accordingly, the medical information control apparatus 10 has caused the authentication apparatus 200 to perform authentication. Further, since facial authentication has been successful, the medical information control apparatus 10 transmits the attribute information of the user U1 to the facial authentication unit 300.

The attribute information display 345 shows the authentication ID of the user U1, the name, sex, and nationality of the user U1, the language he/she uses, allergy information, medications he/she is taking, medical history, and information on the insurance purchased by the user. The attribute information shown in FIG. 15 is merely one example and may display, in addition to or in place of the aforementioned information, other information items. In this way, the medical information control system 700 outputs the attribute information of the user related to the facial authentication to the medical institution or the like. The medical institution or the like is able to refer to the acquired attribute information when this medical institution or the like provides the medical practice, the medical examination, and the drug preparation for the user. That is, the medical information control system 700 helps to support the medical practice, the medical examination, and the drug preparation for the user in the medical institution or the like by outputting the attribute information of the user related to the facial authentication to the facial authentication unit 300.

While the fourth example embodiment has been described above, the medical information control system 700 according to the fourth example embodiment may configure the medical information control apparatus 20 or the medical information control apparatus 30 described above in place of the medical information control apparatus 10.

The aforementioned program can be stored and provided to a computer using any type of non-transitory computer readable media. Non-transitory computer readable media include any type of tangible storage media. Examples of non-transitory computer readable media include magnetic storage media (e.g., flexible disks, magnetic tapes, hard disk drives), optical magnetic storage media (e.g., magneto-optical disks), CD-Read Only Memory (ROM), CD-R, CD-R/W, semiconductor memories (e.g., a mask ROM, a Programmable ROM (PROM), an Erasable PROM (EPROM), a flash ROM, or a Random Access Memory (RAM)). Further, the program(s) may be provided to a computer using any type of transitory computer readable media. Examples of transitory computer readable media include electric signals, optical signals, and electromagnetic waves. Transitory computer readable media can provide the program to a computer via a wired communication line (e.g., electric wires, and optical fibers) or a wireless communication line.

Note that the present invention is not limited to the aforementioned example embodiments and may be changed as appropriate without departing from the spirit of the present invention.

The whole or part of the example embodiments disclosed above can be described as, but not limited to, the following supplementary notes.

### (Supplementary Note 1)

A medical information control apparatus comprising:

image data acquisition means for acquiring image data including a face image of a user captured by a predetermined image-capturing apparatus;

authentication control means for causing an authentication apparatus that stores face feature information of the user to perform facial authentication regarding the image data;

attribute information acquisition means for acquiring, when the facial authentication has been successful, attribute information of the user related to the facial authentication from a storage apparatus that stores the attribute information of the user including information on at least one of a medical practice, a medical examination, and a drug preparation for the user; and

output means for outputting the attribute information associated with the user to a predetermined display terminal that corresponds to the image-capturing apparatus.

### (Supplementary Note 2)

The medical information control apparatus according to Supplementary Note 1, wherein

the image data acquisition means acquires administrator information regarding the administrator of the image-capturing apparatus along with the image data, and

the attribute information acquisition means acquires the attribute information based on the administrator information from the storage apparatus.

### (Supplementary Note 3)

The medical information control apparatus according to Supplementary Note 1 or 2, wherein

the image data acquisition means acquires administrator information regarding the administrator of the image-capturing apparatus along with the image data, and

the attribute information acquisition means outputs the attribute information based on the administrator information to the display terminal.

### (Supplementary Note 4)

The medical information control apparatus according to any one of Supplementary Notes 1 to 3, further comprising reception means for receiving input of a predetermined signal from the display terminal,

wherein the output means outputs the attribute information to the display terminal based on the signal.

### (Supplementary Note 5)

The medical information control apparatus according to Supplementary Note 4, wherein

the reception means receives a condition for extracting the attribute information, and

the output means extracts predetermined information included in the attribute information based on the signal and outputs the extracted information to the display terminal.

### (Supplementary Note 6)

The medical information control apparatus according to Supplementary Note 4, wherein

the reception means receives an inquiry related to at least one of the medical practice, the medical examination, and the drug preparation for the user, and

the output means outputs a response to the inquiry based on the attribute information.

### (Supplementary Note 7)

The medical information control apparatus according to Supplementary Note 6,

further comprising determination means for generating a result of a determination regarding a combination of the inquiry with the attribute information, wherein

the reception means receives a determination request signal for requesting the result of the determination, and

the output means outputs the result of the determination that corresponds to the request to the display terminal.

### (Supplementary Note 8)

The medical information control apparatus according to any one of Supplementary Notes 4 to 7, further comprising update means for updating, when a signal for updating the attribute information has been received from the reception means, the attribute information and causing the storage apparatus to store the updated attribute information.

### (Supplementary Note 9)

The medical information control apparatus according to any one of Supplementary Notes 4 to 8, wherein

the reception means receives a message to be sent to the user, and

the output means sends the message to the user based on an access method included in the attribute information.

### (Supplementary Note 10)

The medical information control apparatus according to Supplementary Note 9, further comprising third party information acquisition means for acquiring third party information related to the message,

wherein the output means sends the message and the third party information related to the message to the user.

### (Supplementary Note 11)

A medical information control system comprising:

the medical information control apparatus according to any one of claims 1 to 10; and

a storage apparatus that stores the user and attribute information of the user in association with each other.

### (Supplementary Note 12)

A medical information control system comprising:

the medical information control apparatus according to any one of claims 1 to 10; and

the authentication apparatus that receives the image data from the medical information control apparatus and performs the facial authentication.

### (Supplementary Note 13)

A medical information control method, wherein a computer executes the processing of:

acquiring image data including a face image of a user captured by a predetermined image-capturing apparatus;

causing an authentication apparatus that stores face feature information of the user to perform facial authentication for the image data;

acquiring, when the facial authentication has been successful, attribute information from a storage apparatus that stores the user and the attribute information of the user in association with each other; and

outputting the attribute information associated with the user to a predetermined display terminal.

### (Supplementary Note 14)

A non-transitory computer readable medium storing a medical information control program for causing a computer to execute the processing of:

acquiring image data including a face image of a user captured by a predetermined image-capturing apparatus;

causing an authentication apparatus that stores face feature information of the user to perform facial authentication for the image data;

acquiring, when the facial authentication has been successful, attribute information from a storage apparatus that stores the user and the attribute information of the user in association with each other; and

outputting the attribute information associated with the user to a predetermined display terminal.

### REFERENCE SIGNS LIST

10 Medical Information Control Apparatus
20 Medical Information Control Apparatus
30 Medical Information Control Apparatus
110 Image Data Acquisition Unit
111 Authentication Control Unit
112 Attribute Information Acquisition Unit
113 Output Unit
114 Receiving Unit
115 Determination Unit
120 Storage Unit
121 Attribute Information
122 Reference Information
130 Buffer Memory
140 Communication Unit
200 Authentication Apparatus
210 Face Feature DB
220 Face Detection Unit
230 Feature Point Extraction Unit
240 Registration Unit
250 Authentication Unit
300 Facial Authentication Unit
301 Image-capturing Apparatus
302 Display Terminal
400 User Terminal
500 Network
700 Medical Information Control System

What is claimed is:

1. A medical information control apparatus comprising:
at least one memory storing instructions, and
at least one processor configured to execute the instructions to;
acquire image data including a face image of a user captured by a predetermined image-capturing apparatus;
cause an authentication apparatus that stores face feature information of the user to perform facial authentication regarding the image data;
acquire, when the facial authentication has been successful, attribute information of the user related to the facial authentication from a storage apparatus that stores the attribute information of the user including information on at least one of a medical practice, a medical examination, and a drug preparation for the user; and
output the attribute information associated with the user to a predetermined display terminal that corresponds to the image-capturing apparatus.

2. The medical information control apparatus according to claim 1, wherein the at least one processor is further configured to execute the instructions to
acquire administrator information regarding the administrator of the image-capturing apparatus along with the image data, and
acquire the attribute information based on the administrator information from the storage apparatus.

3. The medical information control apparatus according to claim 1, wherein
the at least one processor is further configured to execute the instructions to
acquire administrator information regarding the administrator of the image-capturing apparatus along with the image data, and
output the attribute information based on the administrator information to the display terminal.

4. The medical information control apparatus according to claim 1, wherein
the at least one processor is further configured to execute the instructions to
receive input of a predetermined signal from the display terminal, and
output the attribute information to the display terminal based on the signal.

5. The medical information control apparatus according to claim 4, wherein
the at least one processor is further configured to execute the instructions to
receive a condition for extracting the attribute information, and
extract predetermined information included in the attribute information based on the signal and outputs the extracted information to the display terminal.

6. The medical information control apparatus according to claim 4, wherein the at least one processor is further configured to execute the instructions to
receive an inquiry related to at least one of the medical practice, the medical examination, and the drug preparation for the user, and
output a response to the inquiry based on the attribute information.

7. The medical information control apparatus according to claim 6,
the at least one processor is further configured to execute the instructions to generate a result of a determination regarding a combination of the inquiry with the attribute information,
receive a determination request signal for requesting the result of the determination, and
output the result of the determination that corresponds to the request to the display terminal.

8. The medical information control apparatus according to claim 4,
the at least one processor is further configured to execute the instructions to
update, when a signal for updating the attribute information has been received, the attribute information, and
cause the storage apparatus to store the updated attribute information.

9. The medical information control apparatus according to claim 4, wherein
the at least one processor is further configured to execute the instructions to
receive a message to be sent to the user, and
send the message to the user based on an access method included in the attribute information.

10. The medical information control apparatus according to claim 9,
the at least one processor is further configured to execute the instructions to
acquire third party information related to the message,
send the message and the third party information related to the message to the user.

11. A medical information control system comprising:
the medical information control apparatus according to claim 1; and
a storage apparatus that stores the user and attribute information of the user in association with each other.

12. A medical information control system comprising:
the medical information control apparatus according to claim 1; and
the authentication apparatus that receives the image data from the medical information control apparatus and performs the facial authentication.

13. A medical information control method, wherein a computer executes the processing of:
acquiring image data including a face image of a user captured by a predetermined image-capturing apparatus;
causing an authentication apparatus that stores face feature information of the user to perform facial authentication for the image data;
acquiring, when the facial authentication has been successful, attribute information from a storage apparatus that stores the user and the attribute information of the user in association with each other; and
outputting the attribute information associated with the user to a predetermined display terminal.

14. A non-transitory computer readable medium storing a medical information control program for causing a computer to execute the processing of:
acquiring image data including a face image of a user captured by a predetermined image-capturing apparatus;
causing an authentication apparatus that stores face feature information of the user to perform facial authentication for the image data;
acquiring, when the facial authentication has been successful, attribute information from a storage apparatus that stores the user and the attribute information of the user in association with each other; and
outputting the attribute information associated with the user to a predetermined display terminal.
